(19) United States
(12) Patent Application Publication
(10) Pub. No.: US 2022/0113263 A1
(43) Pub. Date: Apr. 14, 2022

(54) X-RAY IMAGING SYSTEM AND METHOD FOR RECOGNIZING FOREIGN OBJECT THEREBY

(71) Applicant: SHIMADZU CORPORATION, KYOTO (JP)

(72) Inventors: Naomasa HOSOMI, KYOTO (JP); Erzhong HU, KYOTO (JP); Tomohiro NAKAYA, KYOTO (JP)

(73) Assignee: SHIMADZU CORPORATION, KYOTO (JP)

(21) Appl. No.: 17/482,222

(22) Filed: Sep. 22, 2021

(30) Foreign Application Priority Data
Oct. 14, 2020 (JP) 2020-173585

(51) Int. Cl. G01N 23/04; G06T 7/00; A61B 6/00

(52) U.S. Cl. CPC G01N 23/04; G06M 11/00; A61B 6/465; G06T 7/0012

(57) ABSTRACT

An X-ray imaging system has an X-ray imaging apparatus include a foreign object image generation element that generates a foreign object image to make sure there is no foreign object left inside a subject body after a surgical operation. A first display unit displays at least one image selected from a group consisting of an X-ray image and a foreign object image. The X-ray imaging system is connected to an X-ray imaging apparatus through a network and displays the foreign object image on the second display unit that provides a better visual recognition level than the first display unit.

### CROSS REFERENCE TO RELATED APPLICATIONS

This application relates to, and claims priority from JP 2020-173585 filed Oct. 14, 2020, the entire contents of which are incorporated herein by reference.

### FIGURE SELECTED FOR PUBLICATION

FIG. 1

### BACKGROUND OF THE INVENTION

### Field of the Invention

The present invention relates to an X-ray imaging system and a method for recognizing a foreign object.

### Description of the Related Art

Conventionally, a gauze counting device that counts the number of used gauze sponges that are used during a surgical operation is known to make sure whether the used gauze sponge is left inside a patient (subject) body or not. Such a gauze counting device is disclosed in JP 2018-48905 A.

The gauze counting device disclosed in JP 2018-48905 A comprises an X-ray generation element that irradiates X-rays to a gauze sponge housing that houses a plurality of gauze sponges in which contrast strings are woven and an X-ray imaging element that images an X-ray fluoroscopic image relative to the gauze sponge housing. The gauze counting device disclosed in JP 2018-48905 A calculates (counts) the number of gauze sponges from the image constructed based on the imaged X-ray fluoroscopic image.

Whereas, when a counting mistake takes place on counting, the results of respective counting numbers before and after the surgical operation may coincide with each other despite leaving a surgical instrument such as used gauze sponge inside the patient (subject) body. Accordingly, not disclosed in JP 2018-48905 A are measures to make sure whether any surgical instrument such as used gauze sponge (foreign object), which is left inside the patient (subject) body, or not, by taking an X-ray image regardless coincidence of counting results before and after the surgical operation would be undertaken.

### PATENT DOCUMENT

1 JP2018-48905 A

Nevertheless, an oversight may take place due to the reason of which a surgical instrument (foreign object) overlaps such as the used gauze sponge and a structure of the human body such as a bone even if it was confirmed that no foreign object of the surgical instrument such as the used gauze sponge is not left inside the patient (subject) body thereof by taking the X-ray image. Therefore, a prevention of an occurrence of the oversight of the foreign object, which is left inside the subject body after the surgical operation, is desirable.

### ASPECTS AND SUMMARY OF THE INVENTION

The present invention relates to addressing at least one of the aforementioned problems, and an object of the present invention is to provide an X-ray imaging system that can prevent an occurrence of an oversight relative to the foreign object left inside the subject body after the surgical operation and a method for recognizing such a foreign object left inside the body of the subject.

An X-ray imaging system according to a first aspect of the present invention comprises an X-ray imaging apparatus further comprising an X-ray irradiation unit that irradiates an X-ray to a subject, a detection unit that detects the X-ray that is irradiated from the X-ray irradiation unit and transmits through the subject, an X-ray image generation unit that generates an X-ray image based on the detection signal that is detected by the X-ray detection unit, a foreign object image generation unit that generates a foreign object image to recognize a foreign object left inside the subject body after a surgical operation based on the X-ray image, and a first display unit that display at least one image selected from a group consisting of the X-ray image and the foreign object image, and a second display unit that displays the foreign object image, wherein the second display unit is installed separately from the X-ray imaging apparatus and connected to the X-ray imaging apparatus via a network, and a visual recognition level of the second display unit is better than a visual recognition level of the first display unit.

A method for recognizing a foreign object according to a second aspect of the present invention comprises a step of irradiating an X-ray to a subject, a step of detecting the X-ray that transmits through the subject; a step of generating an X-ray image based on detection signals detected thereby, a step of generating a foreign object image to recognize a foreign object left inside the subject body after a surgical operation based on the X-ray image, and a step of displaying the foreign object image on the second display unit, wherein the second display unit is installed separately from the X-ray imaging apparatus and connected to the X-ray imaging apparatus via a network, and a visual recognition level of the second display unit is better than a visual recognition level of the first display unit.

According to one alternative aspect of the present invention, with regard to the X-ray imaging system according to the first aspect and the method for recognizing the foreign object according to the second aspect, the foreign object image to recognize the foreign object left inside the subject body after the surgical operation is generated. And the second display unit that is installed separately from the X-ray imaging apparatus and provides the visual recognition level that is better than the visual recognition level of the first display unit, which the X-ray imaging apparatus is so equipped with, displays the foreign object image. Therefore, a user can visually recognize the foreign object image applied to recognize the foreign object left inside the subject body after the surgical operation on the second display unit that has better visual recognition level than the first display unit which the X-ray imaging apparatus equips with. As a result, comparing with the case when the user recognizes the foreign object on the first display unit, which the X-ray imaging apparatus is so equipped, the user can easily recognize the foreign object in the foreign object image, so that the present invention can provide the X-ray imaging system that can prevent an occurrence of an oversight as to the foreign object left inside the subject body after the surgical operation and the method for recognizing such a foreign object left inside the subject body.

The above and other aspects, features and advantages of the present invention will become apparent from the following description read in conjunction with the accompanying drawings, in which like reference numerals designate the same elements.

### BRIEF DESCRIPTION OF THE DRAWINGS

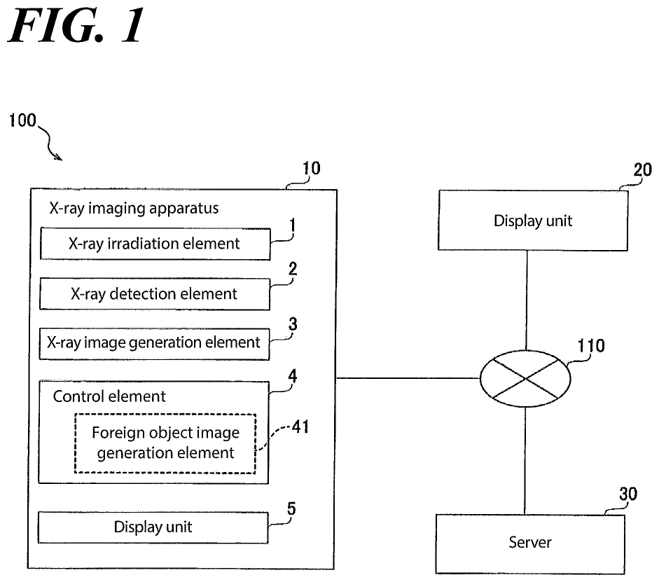

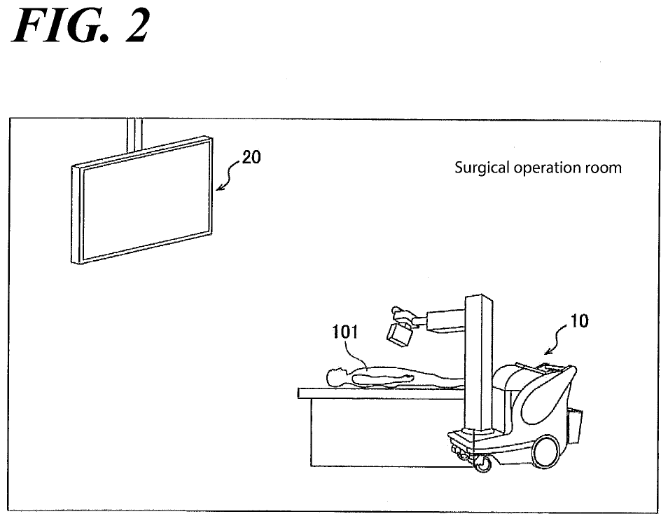

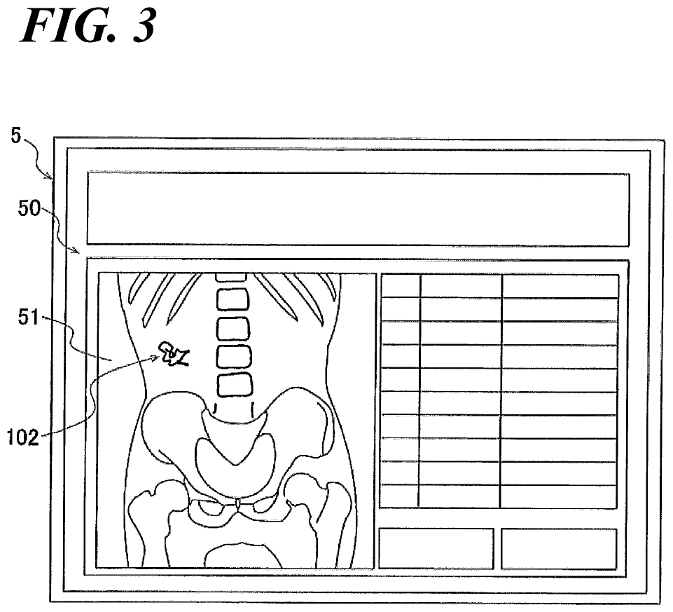

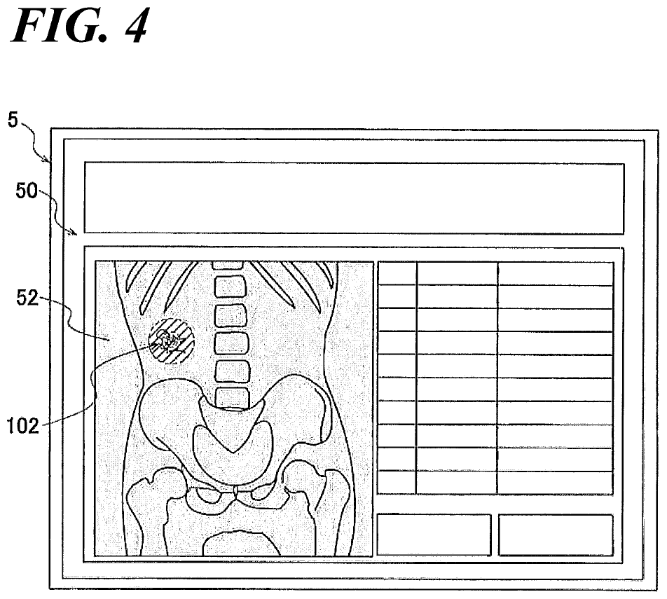

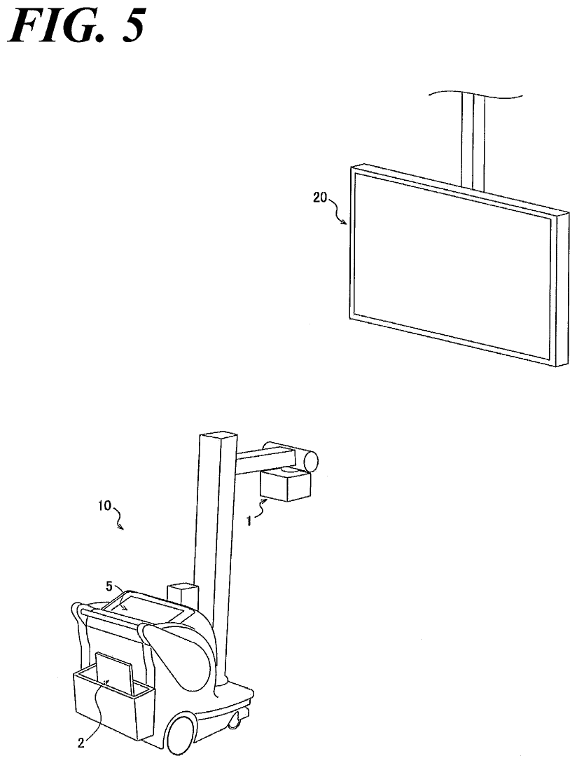

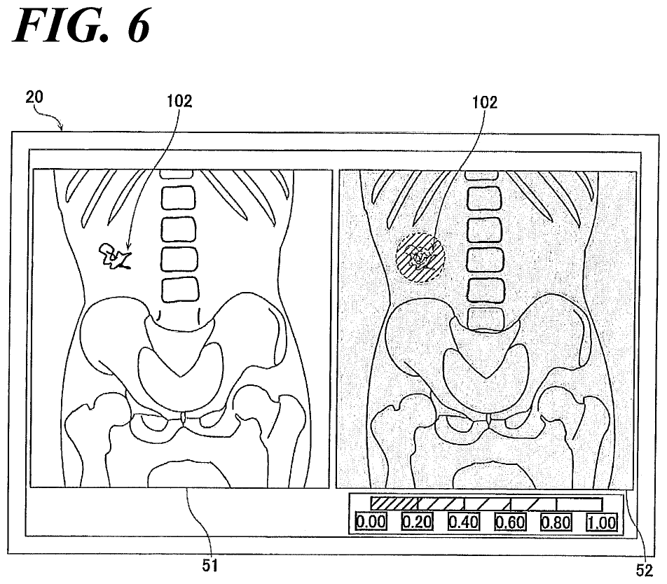

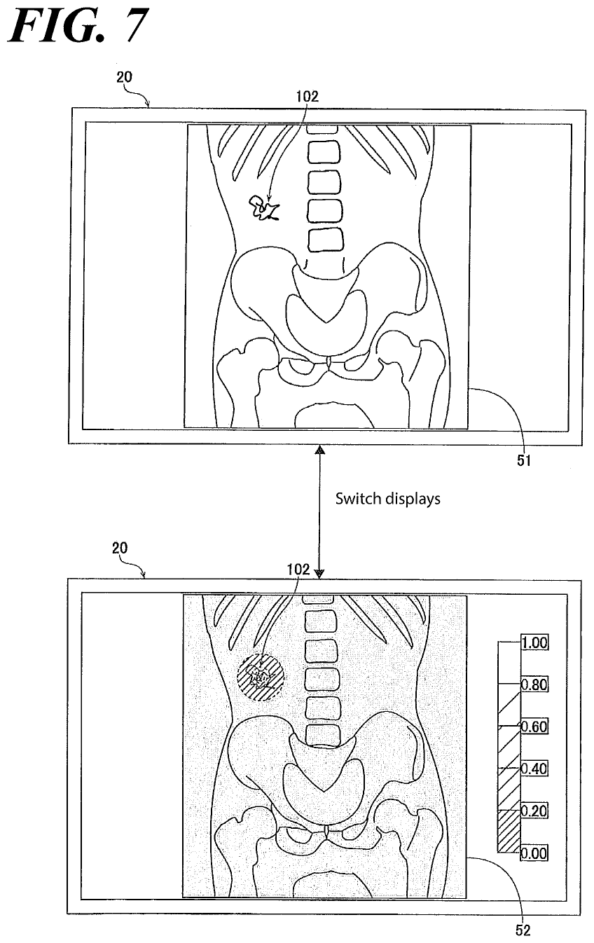

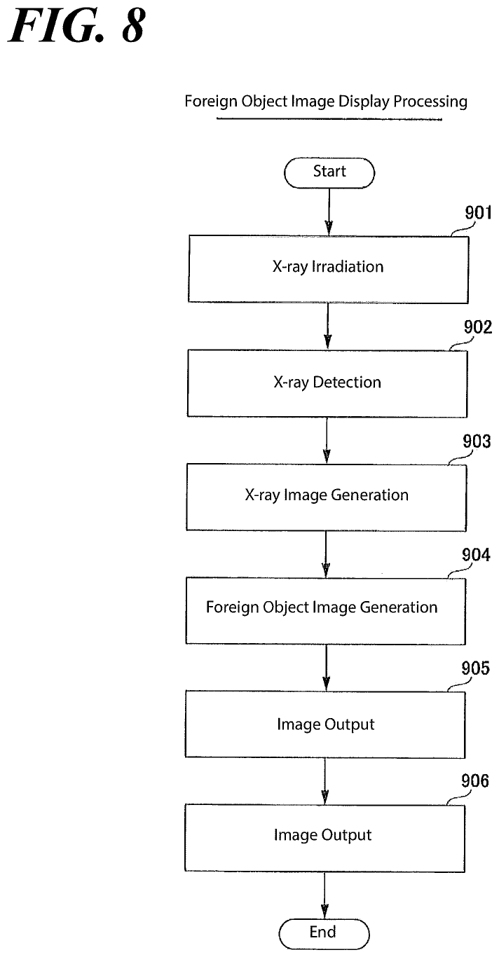

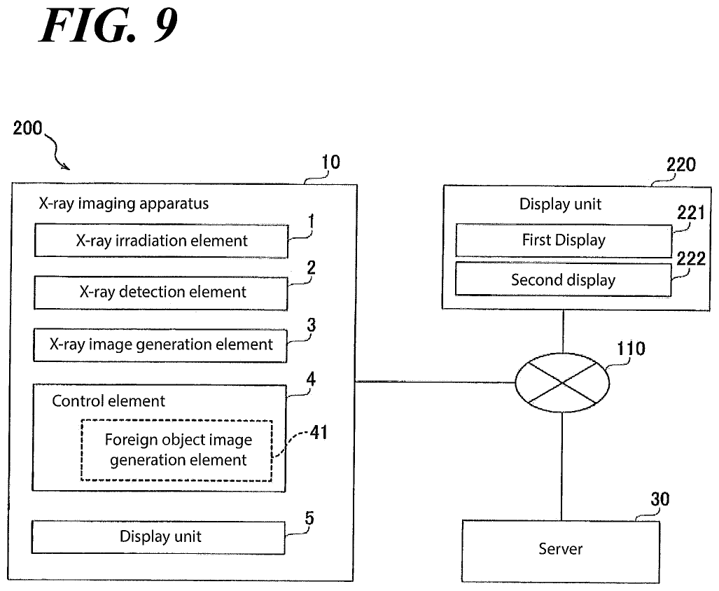

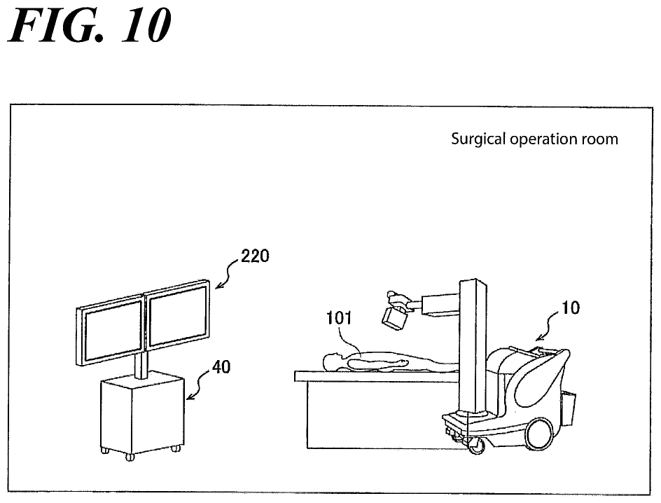

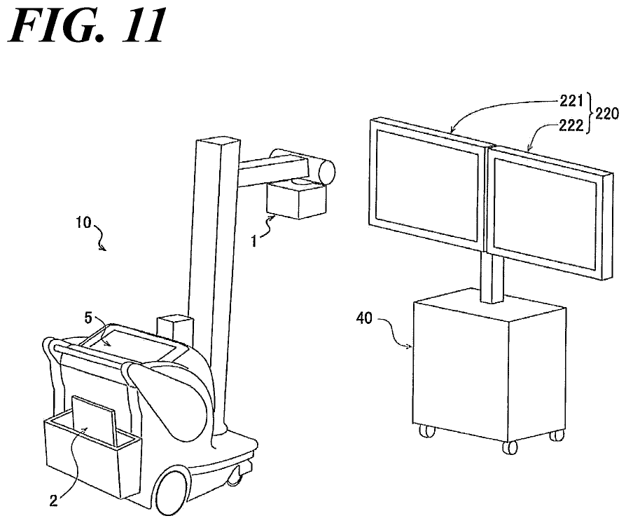

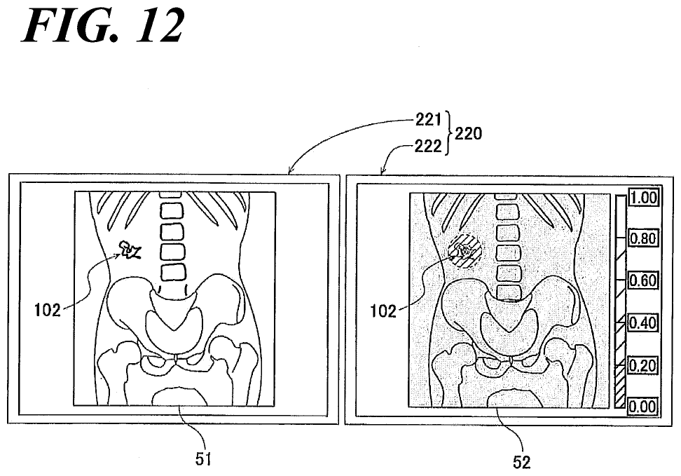

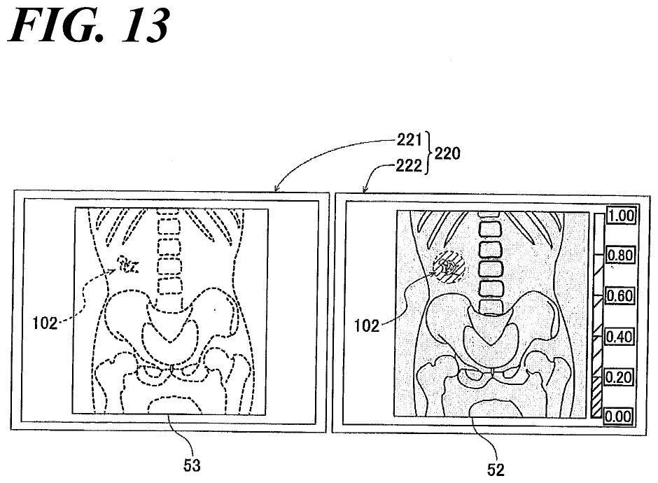

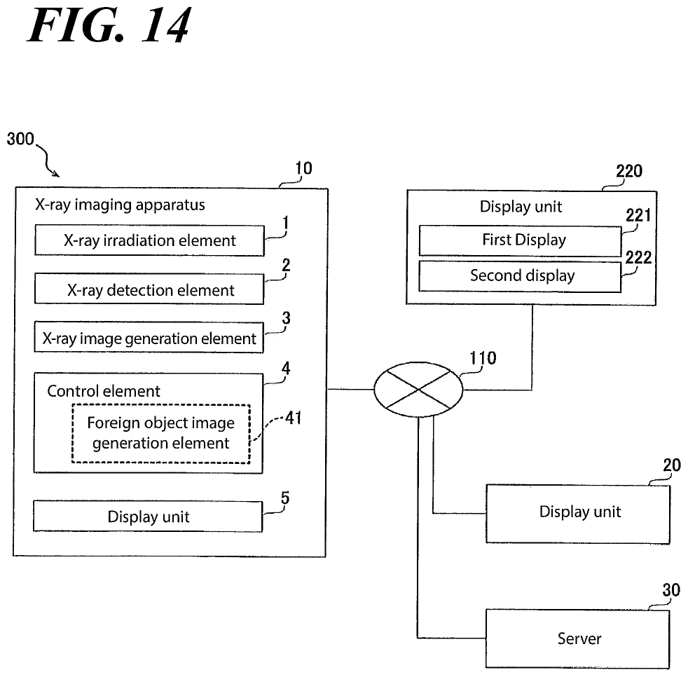

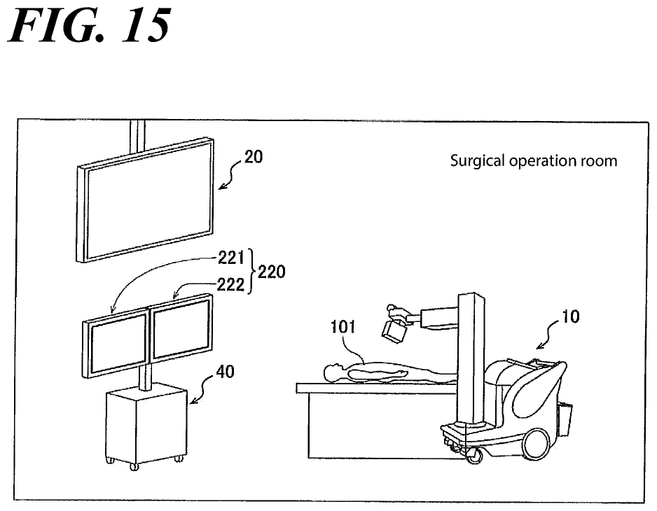

FIG. 1 is a block diagram illustrating the entire structure of the X-ray imaging system of Embodiment 1 of the present invention.

FIG. 2 is a view illustrating the X-ray imaging apparatus and the display unit installed separately from the X-ray imaging apparatus in the surgical operation room according to Embodiment 1 of the present invention.

FIG. 3 is a view illustrating the first figure that is a display example displayed on the display unit of the X-ray imaging apparatus according to Embodiment 1 of the present invention.

FIG. 4 is a view illustrating the second figure that is another display example displayed on the display unit of the X-ray imaging apparatus of Embodiment 1 of the present invention.

FIG. 5 is a view illustrating the display unit of the X-ray imaging apparatus of Embodiment 1 of the present invention, and the display unit installed separately from the X-ray imaging apparatus.

FIG. 6 is a view illustrating the first figure that is a display example displayed on the display unit that is installed separately from the X-ray imaging apparatus of Embodiment 1 of the present invention.

FIG. 7 is a view illustrating the second figure that is another display example displayed on the display unit that is installed separately from the X-ray imaging apparatus of Embodiment 1 of the present invention.

FIG. 8 is a flow chart illustrating a control processing relative to a display method of the foreign object image according to the X-ray imaging system of Embodiment 1 of the present invention.

FIG. 9 is a block diagram illustrating the entire structure of the X-ray imaging of Embodiment 2 of the present invention.

FIG. 10 is a view illustrating the X-ray imaging apparatus and the display unit installed separately from the X-ray imaging apparatus in the surgical operation room of Embodiment 2 of the present invention.

FIG. 11 is a view illustrating the display unit of the X-ray imaging apparatus of Embodiment 2 of the present invention, and the display unit installed separately from the X-ray imaging apparatus.

FIG. 12 is a view illustrating the first figure that is a display example displayed on the first display unit and the second display unit of Embodiment 2 of the present invention.

FIG. 13 is a view illustrating the second figure that is another display example displayed on the first display and the second display of Embodiment 2 of the present invention.

FIG. 14 is a block diagram illustrating the entire structure of the X-ray imaging system of Embodiment 3 of the present invention.

FIG. 15 is a view illustrating the X-ray imaging apparatus and the display unit installed separately from the X-ray imaging apparatus in the surgical operation room of Embodiment 3 of the present invention.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’, ‘link’, ‘connect’, and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

### Embodiment 1

Referring to FIG. 1 to FIG. 8, the inventors illustrate the structure of the X-ray imaging system 100 according to Embodiment 1 of the present invention.

The X-ray imaging system 100 (referring to FIG. 1) comprises the X-ray imaging apparatus 10 (referring to FIG. 1 and FIG. 2) and is configured to display the X-ray image 51 (referring to FIG. 3) or the foreign object image 52 (referring to FIG. 4) on the display unit 20 (referring to FIG. 1 and FIG. 2) that is installed separately from the X-ray imaging apparatus 10. The X-ray imaging system 100 is applied to make sure whether any foreign object 102 (retained foreign object) that is left inside the subject body 101 (referring to FIG. 2) exists or not.

The X-ray imaging system 100 comprises a server 30 (referring to FIG. 1). The server 30 is a server that stores the image data such as DICOM (Digital Imaging and Communications in Medicine) server, which and configured to have DICOM transmitting function. The server 30 receives the image data sent from a variety of examination instruments and stores the data in a database. Further, the image stored in the server 30 is forwarded to the display terminal following the user request. Further, the X-ray imaging apparatus 10, the display unit 20 and server 30 are respectively connected through the network 110.

The X-ray imaging system 100 uses the system that receives the image data from a variety of examination instruments in the hospital, stores the data in the database and displays the data on the display terminal unit. For an example, the X-ray imaging system 100 uses PACS (Picture Archiving and Communication Systems: Image Storing Communication System). Further, the X-ray imaging system 100 may further include a variety of data systems such as RIS (Radiology Information Systems: Radiology Department Information System) as to the examination using radiology examination instrument and HIS (Hospital Information System) that manages an appointment for a medical treatment and the examination results.

The X-ray imaging apparatus 10 is a mobile (portable) X-ray imaging apparatus designed as capable of moving around. The X-ray imaging apparatus 10 is carried into the surgical operation room after the surgical operation such as an open surgery. The X-ray imaging apparatus 10 as to the X-ray imaging system 100 is the instrument to perform the X-ray imaging to make sure whether any foreign object 102 that is left inside the subject body 101 exists or not. In addition, according to Embodiment 1 of the present invention, the X-ray imaging apparatus 10 performs the X-ray imaging, generates the X-ray image 51 and the foreign object image 52, and displays the foreign object image 52 on the display unit 20, regardless of that the number of surgical instruments counted before the surgical operation coincides with the number thereof after the surgical operation.

### [Structure of an X-Ray Imaging Apparatus]

Referring to FIG. 1, the X-ray imaging apparatus 10 comprises an X-ray irradiation element 1, an X-ray detection element 2, an X-ray image generation element 3, and a control element 4 and the display unit 5. In addition, the control element 4 further comprises a foreign object image generation element 41. In addition, the display unit 5 is an example of the first display unit in Claims of the present invention.

The X-ray irradiation element 1 irradiates X-ray toward the subject 101. The X-ray irradiation element 1 further comprises an X-ray tube (not shown in FIG.) that irradiates X-ray when the voltage is added.

The X-ray detection element 2 detects the X-ray that is irradiated from the X-ray tube 1 and then transmits the subject 101. Then, the X-ray detection element 2 outputs a detection signal based on the detected X-ray. The X-ray detection element 2 includes e.g., a FPD (Flat Panel Detector). The X-ray detection element 2 is a wireless X-ray detector and outputs the detection signal as the wireless signal. Specifically, the X-ray detection element 2 connects wirelessly and communicably with the X-ray image generation element 3, later described, through a wireless LAN (Local Area Network) and outputs the detection signal for the X-ray image generation element 3 as the wireless signal.

The X-ray image generation element 3 generates the X-ray images 51 (referring to FIG. 3) based on the detection signal that the X-ray detection element 2 detects. The X-ray image generation element 3 connects wirelessly and communicably with the X-ray detection element 2. The X-ray image generates element 3 comprises a processor such as a FPGA (Field-programmable Gate Array) designed for an image processing. And the image generation element 3 outputs the generated X-ray images 51 to a control element 4, later described. In addition, the X-ray image 51 is the image of the subject (patient) 101 imaged using X-ray after the surgical operation.

The control element 4 is a computer comprising such as a CPU (Central Processing Unit), ROM (Read Only Memory) and RAM (Random Access Memory). The control element 4 further comprises the foreign object image generation element 41. Specifically, the control element 4 is functionally operative as the foreign object image generation element 41 while executing a predetermined control program. In addition, the foreign object image generation element 41 is a function block as a software in the control element 4 and functions based on a command signal from the control element 4. The control element 4 executes controls of such as the X-ray imaging apparatus 10 and the data communication between the external instruments (outputs of the X-ray image 51 and the foreign object image 52).

The foreign object image generation element 41 generates the foreign object image 52 for recognizing the foreign object 102 left inside the subject body 101 after the surgical operation based on the X-ray image 51.

Further, the foreign object 102 left inside the subject body 101 is at least one material selected from a group consisting of a gauze sponge, a suture needle, and a forceps. In addition, X-ray impermeant matter such as barium sulphate, which blocks the X-ray, is woven in the gauze sponge as an X-ray contrast thread. Accordingly, the gauze sponge is incorporated (imaged) in the X-ray image 51 with the X-ray imaging after the surgical operation. Specifically, the gauze sponge can be imaged using the X-ray imaging apparatus 10. According to the aspect of Embodiment 1, the foreign object 102 includes the gauze sponge left inside the subject body 101 after the surgical operation.

The foreign object image generation element 41 generates the foreign object image 52 while executing an image processing to enhance the foreign object 102 left inside the subject body 101 after the surgical operation. Specifically, according to Embodiment 1, the foreign object image 52 (referring to FIG. 4) is a foreign object enhanced image in which the foreign object 102 left inside the subject body 101 after the surgical operation is enhanced.

The image processing using the foreign object generation element 41 (generation of the foreign object image 52) is a processing to support the user to recognize the existence of the foreign object 102 by eyes. The image processing using the foreign object generation element 41 (generation of the foreign object image 52) comprises a change processing of the brightness value of the image, the processing for enhancing the characteristics structure of the foreign object 102 in the image, or the processing for providing the location and shape of the foreign object 102 in the image.

According to Embodiment 1, the foreign object image generation element 41 detects the foreign object 102 and generates the foreign object image 52 while executing the image processing for enhancing the foreign object 102 left inside the subject body 101 after the surgical operation based on the learned model created by a machine learning. The foreign object generation element 41 (control element 4) generates the foreign object image 52 to recognize (identify) the foreign object 102 while coloring the portion corresponding to the region including the detected foreign object 102.

The foreign object generation element 41 (control element 4) acquires an estimated region in which the foreign object 102 is included in the X-ray image 51 and also acquires a decided value by which the likelihood (reliability), which indicates the certainty level as to the foreign object 102 in the estimated region, is decided based on the X-ray image 51 and the learned model. And the foreign object generation element 41 (control element 4) generates the foreign object image 52 as a colored heat map image (color map image) in which respective colors change corresponding to the decided value. And according to Embodiment 1 and referring to FIG. 4, the foreign object generation element 41 (control element 4) generates the foreign object image 52 including the distribution display colored following the 2-dimensional Gaussian distribution every acquired estimated regions based on the size (area) and the decided value of the estimated region.

Further, the foreign object generation element 41 (control element 4) recognizes (identifies) the linear structure of the foreign object 102 and may generates the foreign object image 52 with coloring based on the recognized linear structure. For example, the foreign object generation element 41 (control element 4) may generates the foreign object image 52 as the heat map image (color map image) colored while varying corresponding to the density of the linear structure of the recognized foreign object 102.

In such a case, the foreign object generation element 41 (control element 4) detects the foreign object 102 in the X-ray image 51 and generates the image from which the foreign object 102 detected in the X-ray image 51 is removed based on the X-ray image 51 and the learned model generated by the machine learning. And the foreign object generation element 41 (control element 4) generates the foreign object image 52 based on the X-ray image 51 and the image from which the detected foreign object 102 is removed, For example, the foreign object generation element 41 (control element 4) acquires the difference between the image from which the detected foreign object 102 is removed and the X-ray image 51, recognizes the linear structure of the foreign object 102 based on the acquired difference, and generates the foreign object image 52 while coloring based on the linear structure of the recognized foreign object 102.

In addition, the image processing (generation of the foreign object image 52) using the foreign object image generation element 41 may use a general image processing without using the learned model generated by the machine learning.

The display unit 5 further comprises such as a liquid crystal display or an organic EL display. The display unit 5 connected with the control element 4 through e.g., a video (image) interface such as HDMI®. Further, Embodiment 1, the display unit 5 is a touch panel display installed to the X-ray imaging apparatus 10 and functions also as an operation unit for receiving a user operation as to the X-ray imaging apparatus 10.

Further, the display unit 5 displays at least one image selected from a group consisting of the X-ray image 51 (referring to FIG. 3) and the foreign object image 52 (referring to FIG. 4).

The display image 50 displayed on the display unit 5 (operation screen) is alternatively selected from the X-ray image 51 or the foreign object image 52 and displayed. Specifically, the display unit 5 can switch the display image between the X-ray image 51 and the foreign object image 52 as for the display image 50.

Such as the information of the imaging protocol for X-ray imaging and the operation buttons as GUI (Graphical User Interface) are displayed other than the X-ray image 51 or the foreign object image 52 in the display image 50 displayed on the display unit 5 (operation screen). In addition, the display unit 5 may display only the X-ray image 51 or the foreign object image 52 based on the operation by the user. The images displayed on the display unit 5 is switchable in accordance with the user setting or operation.

The X-ray imaging apparatus 10 performs controls for the X-ray imaging and generations of the X-ray image 51 and the foreign object image 52 based on the imaging protocol.

The imaging protocol includes such as the information of the subject (patient) 101, the imaging condition (such as an imaging region and a radiation exposure), and the operative procedure for the surgical operation. Further, the imaging protocol may include the display source of the X-ray image 51 and the foreign object image 52.

The X-ray imaging apparatus 10 incorporates a part of the imaging protocol or all information while reading the bar code of the list band or the RF (radio frequency) tag worn by the subject (patient) 101.

(Structure of the Display Unit Installed Separately from the X-Ray Imaging Apparatus 10)

According to Embodiment 1, the X-ray imaging system 100 displays the foreign object image 52 on the display unit 20 (referring to FIG. 5) that is installed separately from the X-ray imaging apparatus 10 and connected to the X-ray imaging apparatus 10 via the network 110 and provides a visual recognition level thereof is better than the display unit 5. In addition, the display unit 20 is an example of the second display unit in Claims.

According to Embodiment 1, the display unit 20 is fixed on the ceiling of the surgical operation room. Specifically, the display unit 20 is a built-in display (monitor) in the surgical operation room.

The display unit 20 is installed in the surgical operation room in advance and provides at least one function selected from a group consisting of the higher image resolution and the larger display area than the display unit 5. According to Embodiment 1, the display unit 20 provides the higher image resolution and the larger display area (screen size) than the display unit 5 thereof. Specifically, the display unit 20 is the larger display capable of providing the higher definition than the display unit 5 thereof.

The display unit 20 displays respectively the X-ray image 51 and the foreign object image 52 at the same time or switches and displays one image selected from a group consisting of the X-ray image 51 and the foreign object image 52 one at a time.

Specifically, according to Embodiment 1 referring to FIG. 6, the display unit 20 displays the X-ray image 51 and the foreign object image 52 at the same time (i.e., simultaneous display). Further, referring to FIG. 7, the display unit 20 can display one of the X-ray image 51 and the foreign object image 52 while switching each other (switching display). Specifically, according to Embodiment 1, a display method for the X-ray image 51 and the foreign object image 52 at the display unit 20 can be switched between the above simultaneous display and the above switching display.

(Aspect of Output and Display of the Foreign Object Image)

The X-ray imaging apparatus 10 outputs the foreign object image 52 to the network 110 after the foreign object image generation element 41 generates the foreign object image 52.

In addition, generation of the foreign object image 52 may be automatically executed after the X-ray image generation element 3 generates the X-ray image 51 or by the user operation after the X-ray image 51 is generated.

Further, the foreign object image 52 is output from the X-ray imaging apparatus 10 to the network 110 based on an operation for completing an imaging relative to the X-ray imaging apparatus 10 With regard to the X-ray imaging system 100, the X-ray image 51 and the foreign object image 52 are output from the X-ray imaging apparatus 10 to the network 110 by that the user performs the operation to complete the imaging as to the X-ray imaging apparatus 10. The X-ray image 51 and the foreign object image 52 output from the X-ray imaging apparatus 10 are saved (stored) in the server 30 via the network 110.

The display unit 20 displays automatically the foreign object image 52 while the foreign object image 52 is forwarded from the network 110 output from the X-ray imaging apparatus 10.

The X-ray image 51 and the foreign object image 52 output from the X-ray imaging apparatus 10 are forwarded to the display unit 20 from the server 30 based on the imaging protocol. Accordingly, the X-ray image 51 and the foreign object image 52 are displayed automatically on the display unit 20 based on the imaging protocol.

Further, the X-ray image 51 and the foreign object image 52 are displayed together with the information for X-ray imaging such as the information of the imaging protocol and the operation buttons as GUI on the display unit 5 of the X-ray imaging apparatus 10 while the X-ray image 51 and the foreign object image 52 are being displayed on the display unit 20 in the surgical operation room. In addition, the images displayed on the display unit 5 is switchable in accordance with the user setting or operation while the display unit 20 in the surgical operation room are displaying the X-ray image 51 and the foreign object image 52.

### Display Method for a Foreign Object of Embodiment 1

Next, referring to FIG. 8, the inventors set forth a processing flow of a method for displaying the foreign object image 52 using the X-ray imaging system 100. In addition, the steps 901 through 905 are controls to be executed by the X-ray imaging apparatus 10, and the step 906 is a control to be executed by the X-ray imaging system 100. In addition, the processing as to the display method for the foreign object image 52 is the processing that starts when the user starts to perform the X-ray imaging by the X-ray imaging apparatus 10 carried into the surgical operation room after the surgery following counting the surgical instrument after the surgical operation regardless coincidence between the number counts of the surgical instrument before and after the surgery. Specifically, the processing as to the display method for the foreign object image 52 is performed after the counting of the surgical instrument, beginning of the surgery, the surgery, closing a surgical incision, and counting the surgical instrument are carried out in order.

First, the X-ray irradiation element irradiates an X-ray at the step 901, The step 901 is the step of irradiating the X-ray from the X-ray irradiation element 1 of the X-ray imaging apparatus 10 to the subject 101 based on the user operation.

Next, the X-ray detection element 2 detects the X-ray at the step 902. The step 902 is the step of detecting the X-ray that transmits through the subject 101 by the X-ray detection element 2 of the X-ray imaging apparatus 10.

Next, the X-ray image 51 is generated at the step 903. The step 903 is the step of generating the X-ray image 51 based on the detected detection signals. The X-ray image generation element 3 of the X-ray imaging apparatus 10 generates the X-ray image 51 based on the detected signals detected by the X-ray image generation element 3 at the step 903.

Next, the X-ray image 52 is generated at the step 904, The step 904 is the step of generating the foreign object image 52 to recognize the foreign object 102 left inside the subject body 101 after the surgery based on the X-ray image 51. The foreign object image generation element 41 of the X-ray imaging apparatus 10 generates the X-ray foreign image 52 based on the X-ray image 51 at the step of 903.

Next, the X-ray image 51 and the foreign object image 52 are output at the step 905, The step 905 is the step of outputting the X-ray image 51 and the foreign object image 52 to the server 30 connected to the network 110 at the step 905, The X-ray image 51 and the foreign object image 52 are output from the X-ray imaging apparatus 10 at the step 905.

Next, the X-ray image 52 is displayed on the display unit 20 at the step 906, The step 906 is the step of displaying the foreign object image 52 on the display unit 20, which is installed separately from the X-ray imaging apparatus 10 having the display unit 5 which displays at least one image from a group consisting of the X-ray image 51 and the foreign object image 52, and connected to the X-ray imaging apparatus 10 through the network 110, and provides the better visual recognition than the display unit 5 thereof.

### Effect of Embodiment 1

The following effects can be obtained of Embodiment 1.

According to Embodiment 1, the X-ray imaging system 100 and the method for recognizing the foreign object generate the foreign object image 52 to recognize the foreign object 102 left inside the subject body 101 after the surgical operation. And the display unit 20 (second display unit) that display the foreign object image 52, which is installed separately from the X-ray imaging apparatus 10 and provides the better visual recognition level than the display unit 5 (first display unit) which the X-ray imaging apparatus 10 equips therewith. Therefore, the user can visually recognize the foreign object image 52 to recognize the foreign object 102 left inside the subject body 101 after the surgical operation on the display unit 20 that provides the better visual recognition level than the display unit 5 which the X-ray imaging apparatus 10 equips therewith. As results, the user easily recognizes the foreign object 102 in the foreign object image 52 by eyes compared with the case of using the display unit 5 of the X-ray imaging apparatus 10, so that an occurrence of an error by oversight of the foreign object 102 left inside the subject body 101 after the surgery can be prevented.

Further, with regard to the X-ray imaging system and the method for recognizing the foreign object according to Embodiment 1, the effect described below can be obtained due to the following structure.

The X-ray imaging system 100 and the method for recognizing the foreign object according to Embodiment 1, the display unit 20 (second display unit) is installed in the surgical operation room in advance and provides the higher level of the screen resolution than the display unit 5 (first display unit) thereof. Therefore, the user can visually recognize the foreign object image 52 for recognizing the foreign object 102 left inside the subject body 101 after the surgical operation on the display unit 20 providing the higher resolution than the display unit 5 which the X-ray imaging apparatus 10 equips therewith, so that the user can easily and securely recognize the foreign object 102 in the foreign object image 52 by eyes compared to the case in which the user should recognize the foreign object 102 on the display unit 5 that the X-ray imaging apparatus 10 equips. As a result, the occurrence of the oversight of the foreign object 102, which is left inside the subject body 101 after the surgical operation, can be prevented. Further, the display unit 20 has the larger display area than the display unit 5. Therefore, the user can visually recognize the foreign object image 52 to recognize the foreign object 102 left inside the subject body 101 after the surgical operation on the display unit 20 having the larger display area than the display unit 5 which the X-ray imaging apparatus 10 equips therewith, so that the user can easily and surely recognize the foreign object 102 in the foreign object image 52 by eyes compared to the case in which the foreign object 102 is recognized in the foreign object image 52 on the display 5 that the X-ray imaging apparatus 10 equips therewith. As a result, the occurrence of the oversight of the foreign object 102, which is left inside the subject body 101 after the surgical operation, can be prevented.

Further, with regard to the X-ray imaging system 100 according to Embodiment 1, the foreign object image generation element 41 generates the foreign object image 52 while executing the image processing to enhance the foreign object 102 left inside the subject body 101 after the surgical operation. Therefore, the foreign object 102 left inside the subject body 101 after the surgical operation is enhanced while (the image processing in which) the foreign object image generation element 41 generates the foreign object image 52. so that the user can further easily recognize the foreign object 102 in the foreign object image 52. As a result, the occurrence of the oversight of the foreign object 102, which is left inside the subject body 101 after the surgical operation, can be further prevented. Further, the foreign object image 52 output from the X-ray imaging apparatus 10 to the network 110 after the foreign object image generation element 41 generates the foreign object image 52 is forwarded from the network 110 and the display unit 41 displays automatically the foreign object image 52. Accordingly, the display unit 20, providing the better visual recognition level than the display unit 5 (first display unit) thereof, which the X-ray imaging apparatus 10 equips therewith, can easily display the foreign object image 52.

Further, with regard to the X-ray imaging system 100 according to Embodiment 1, the foreign object image 52 is output from the X-ray imaging apparatus 10 to the network 110 based on an operation for completing the imaging relative to the X-ray imaging apparatus 10 Accordingly, the foreign object image 52 is automatically output from the X-ray imaging apparatus 10 to the network 110 while the user performs the operation for completing the imaging, so that an exclusive operation to display the foreign object image 52 on the display unit 20 (second display unit) is not required. As a result, the user's workload can be lowered.

Further, with regard to the X-ray imaging system 100 according to Embodiment 1, the display unit 20 (second display unit) is fixed to the ceiling of the surgical operation room and displays respectively the X-ray image 51 and the foreign object image 52 at the same time or switches the display image between the X-ray image 51 and the foreign object image 52 one at a time. Accordingly, the user can recognize the X-ray image 51 and the foreign object image 52 by eyes at the same time when the display unit 20 displays the X-ray image 51 and the foreign object image 52 at the same time, so that the user can compare easily the X-ray image 51 and the foreign object image 52. As a result, the user can easily recognize by eyes whether the foreign object 102 is left inside the subject body or not, so that the occurrence of the oversight of the foreign object 102, which is left inside the subject body 101 after the surgical operation, can be further prevented. Further, the display unit 20 is capable of switching and displaying the image between the X-ray image 51 and the foreign object image 52 even when the display unit 20 displays the image switched between the X-ray image 51 and the foreign object image 52, so that the user can compare easily the X-ray image 51 and the foreign object image 52. As a result, the user can easily recognize by eyes whether the foreign object 102 is left or not, so that the occurrence of the oversight of the foreign object 102, which is left inside the subject body 101 after the surgical operation, can be further prevented.

Further, with regard to the X-ray imaging system 100 according to Embodiment 1, the foreign object 102 left inside the subject body 101 includes at least one material selected from a group consisting of the gauze sponge, the suture needle, and the forceps. Accordingly, the user can visually recognize the foreign object image 52 to recognize the foreign object 102 left inside the subject body 101 after the surgical operation on the display unit 20 (second display unit) providing the better visual recognition level than the display unit 5 thereof with which the X-ray imaging apparatus 10 equips, so that the user can easily recognize the gauze sponge, the suture needle, or the forceps in the foreign object image 52 by eyes compared to the case in which the user recognizes the gauze sponge, the suture needle, or the forceps in the foreign object image 52 on the display 5 (first display) that the X-ray imaging apparatus 10 equips therewith. As a result, the occurrence of the oversight of the gauze sponge, the suture needle, or the forceps, which is left inside the subject body 101 after the surgical operation, can be prevented.

### Embodiment 2

Referring to FIG. 9 to FIG. 14, the inventors illustrate the structure of the X-ray imaging system 200 (referring to FIG. 9) according to Embodiment 2 of the present invention. In addition, the same element as illustrated according to Embodiment 1 has the same sign in each FIG.

According to Embodiment 2, the display unit 220 (referring to FIG. 9 and FIG. 10) installed to the wheeled platform monitor 40 (referring to FIG. 10), of which structure is different from Embodiment 1 in which the display unit 20 which is the display (monitor) fixed in the surgical operation room, displays the foreign object image 52.

The wheeled platform monitor 40 is preliminarily parked in the surgical operation room and movable while having the display unit 220. The wheeled platform monitor 40 is a wheeled platform equipped with the display (monitor) on which the image taken using a C-arm type X-ray imaging apparatus (not shown in FIG.) for the surgical operation is displayed.

Referring to FIG. 11, the display unit 220 further comprises such as a first display 221 and a second display 222. The first display 221 and the second display 222 are in place side by side as to the wheeled platform monitor 40. The first display 221 and the second display 222 further comprises such as a liquid crystal display or an organic EL display.

The display unit 220 provides at least one function selected from a group consisting of the higher image screen resolution and the larger display area than the display unit 5 thereof. According to Embodiment 2, both the first display 221 and the second display 222 provide the higher screen resolution than the display unit 5 thereof. And the total display area (screen size) made by combining the display area of the first display 221 and the display are of the second display 222 is larger than the display area of the display unit 5.

Further, both the first display 221 and the second display 222 provide respectively the higher image resolution than the display unit 5 thereof and have the larger display area (screen size) than the display unit 5 thereof. Specifically, both the first display 221 and the second display 222 provide respectively the higher definition and have the larger display area than the display unit 5 thereof.

The X-ray imaging system 200 displays the foreign object image 52 on the display unit 220 that is installed separately from the X-ray imaging apparatus 10 and connected to the X-ray imaging apparatus 10 via the network 110 and has a better visual recognition level than the display unit 5 thereof. Further, the X-ray imaging apparatus and the display unit 220 are connected to the server 30.

The X-ray imaging system 200 displays the foreign object image 52 on one (e.g., the second display 222) of the first display 221 and the second display 222 and the X-ray image 51 on the other (e.g., the first display 222) (referring to FIG. 12).

Further, the X-ray imaging system 200 displays the foreign object image 52 on one display (e.g., the second display 222) of the first display 221 and the second display 222 and the X-ray fluoroscopic image 53, which is the X-ray fluoroscopy as for the current state of the subject 101, on the other display (e.g., the first display 222) (referring to FIG. 13).

In addition, the X-ray fluoroscopic image 53 is the image taken using the C-arm type X-ray image apparatus (not shown in FIG.) for the surgical operation Specifically, the first display 221 of the display unit 220 displays the X-ray fluoroscopic image 53 taken using the C-arm type X-ray image apparatus (not shown in FIG.) for the surgical operation, and the second display 222 of the display unit 220 displays the foreign object image 52 output from the X-ray imaging apparatus 10.

According to Embodiment 2, the X-ray imaging system 200 is capable of switching the display image displayed on the other display (first display 221) between the X-ray image 51 and the X-ray fluoroscopic image 53.

In addition, the structure according to Embodiment 2 is the same as the aspect of Embodiment 1.

### Effect According to Embodiment 2

The following effects can be obtained according to Embodiment 2.

With regard to the X-ray imaging system 200 and the method for recognizing the foreign object according to Embodiment 2, the occurrence of the oversight of the foreign object 102, which is left inside the subject body 101 after the surgical operation, can be prevented.

Further, with regard to the X-ray imaging system 200 according to Embodiment 2, the effect below can be obtained due to the following structure.

With regard to the X-ray imaging system 200 according to Embodiment 2, the display unit 220 (second display unit) further comprises the first display 221 and the second display 222 positioned side by side on the wheeled platform monitor 40 which is parked in advance in the surgical operation room. And the foreign object image 52 is displayed on one display (e.g., the second display 222) of the first display 221 and the second display 222, and also the X-ray image 51 is displayed on the other display (e.g., the first display 221), so that the X-ray image 51 and the foreign object image 52 can be displayed side by side. As a result, the user can easily compare the X-ray image 51 and the foreign object image 52, so that the occurrence of the oversight of the foreign object 102, which is left inside the subject body 101 after the surgical operation, can be further prevented.

Further, the foreign object image 52 is displayed on one display (e.g., the second display 222) of the first display unit 221 and the second display unit 222 and also the X-ray fluoroscopic image 53, which is the X-ray fluoroscopy as for the current state of the subject 101, is displayed on the other display (e.g., the first display 222), so that the X-ray fluoroscopic image 53 (the current state of the subject 101) and the foreign object image 52 can be displayed side by side. As a result, the user can make sure the current state of the subject 101 using the fluoroscopic image 53 while affirming the location of the foreign object 102 based on the foreign object image 52. so that the recovery of the foreign object 102 (i.e., a repeat surgery) can be swiftly proceeded even when the foreign object 102 is left inside the subject body 101 after the surgical operation, can be further prevented.

In addition, the other effect according to Embodiment 2 is the same as the aspect of Embodiment 1.

### Embodiment 3

Referring to FIG. 14 and FIG. 15, the inventors illustrate the structure of the X-ray imaging system 300 according to Embodiment 3 of the present invention. In addition, the same element as illustrated according to Embodiment 1 and Embodiment 2 has the same sign in FIG.

With regard to the X-ray imaging system 300 (referring to FIG. 14) according to t of Embodiment 3, the display unit 20 (referring to FIG. 14 and FIG. 15) installed and positioned in the surgical operation room, the display unit 220 installed on the wheeled platform monitor 40 parked therein in advance (referring to FIG. 14 and FIG. 15) can display the foreign object image 52. Specifically, with regard to the X-ray imaging system 300 according to Embodiment 3, any one of the display unit 20, the first display 221 and the second display 222 is capable of displaying the foreign object image 52 generated by the foreign object generation element 41.

In addition, the other structure and effect according to Embodiment 3 are the same as the aspect of Embodiment 1 and Embodiment 2.

### Alternative Embodiments

In addition, the alternative aspects of the Embodiments disclosed at this time are examples and not limited thereto in any points. The scope of the present invention is specified in the claims but not in the above description of the aspect of Embodiments and all alternative (alternative examples) are included in the scope of the claims and equivalents thereof.

For example, according to Embodiments 1 to 3 as set forth above, the display unit 20 and 220 (second display unit) provide the higher image resolution and have the larger display areas than the display unit 5 (first display unit) thereof, but the present invention is not limited thereto. According to the present invention, the second display unit may just provide the better screen resolution than the first display unit thereof and just have the larger display area than the first display unit thereof.

Further, according to Embodiments 1 to 3 as set forth above, the foreign object image 52 output from the X-ray imaging apparatus 10 is forwarded from the network 110 and thereby the foreign object image 52 is automatically displayed on the display unit 20 and the display unit 220 (second display unit), but the present invention is not limited thereto. According to the present invention, the foreign object image can be displayed on the second display unit based on the operation by the user.

Further, according to Embodiments 1 to 3 as set forth above, the foreign object image 52 is output from the X-ray imaging apparatus 10 to the network 110 based on an operation for completing an imaging as to the X-ray imaging apparatus 10, but the present invention is not limited thereto. According to the present invention, the foreign object image can be output from the X-ray imaging apparatus to the network after an operation for completing an imaging as to the X-ray imaging apparatus is finished.

Further, according to Embodiment 2 as set forth above, the wheeled platform monitor 40 and the display unit 220 (second display unit) are prepared in the surgical operation room in advance, but the present invention is not limited thereto. According to the present invention, the wheeled platform monitor and the second display unit can be carried into the surgical operation room when the foreign object (retained foreign object) left inside the subject body is being recognized whether the foreign object exists or not after the surgical operation.

Further, according to Embodiment 2 as set forth above, the foreign object image 52 is displayed on one display (e.g., the first display) selected from a group consisting of the first display 221 and the second display 222, and the other rest display (second display 222) switches the display image between the X-ray image 51 and the X-ray fluoroscopic image 53, which is the X-ray fluoroscopy as for the current state of the subject 101, but such a display is not limited thereto. According to the present invention, the foreign object image is displayed on one display selected from a group consisting of the first display and the second display, and on the other display, only one image selected from a group consisting of the X-ray image and the X-ray fluoroscopic image that is the X-ray fluoroscopy as for the current state of the subject may be displayed.

Further, according to Embodiments 1 and 3 as set forth above, the display unit 20 (second display unit) is fixed on the ceiling in the surgical operation room, but the present invention is not limited to such an example. According to the present invention, the second display unit may be fixed on the wall of the surgical operation room.

Further, according to Embodiment 1 to 3 as set forth above, the foreign object 102 left inside the subject body 101 is at least one material selected from a group consisting of a gauze sponge, a suture needle, and a forceps, but the present invention is not limited thereto. According to the present invention, the foreign object may be all of the surgical gauze sponges, the suture needle, and the forceps, or any combination thereof. Further, according to the present invention, the foreign object may be another surgical instrument such as a surgical bolt and a clip for fixing.

In addition, according to Embodiments 1 described above. for convenience of explanation, the inventors set forth a processing of the control processing as to a display method of the foreign object image the present invention is processed in order following the process flow using the flow driving flow chart, but the present invention is not limited thereto. According to the present invention, the processing operation can be performed using an invent driving processing (invent driven processing) every event. In such case, a perfect event driven processing can be applied, or a combination of the event driven processing and flow driven processing can be applied.

### Aspect of the Present Invention

The above examples of the aspects of Embodiments are specific examples in accordance with the below aspects.

(Term 1)

An X-ray imaging system comprises an X-ray imaging apparatus further comprising an X-ray irradiation element that irradiates an X-ray relative a subject, an X-ray detection element that detects the X-ray that is irradiated from the X-ray irradiation element and transmits through the subject, an X-ray image generation element that generates an X-ray image based on an X-ray signal detected by the X-ray detection element, a foreign object image generation element that generates a foreign object image to recognize a foreign object left inside a subject body after a surgical operation based on the X-ray image, and a first display unit that displays at least one image selected from a group consisting of the X-ray image and the foreign object image, and a second display unit that displays the foreign object image, wherein the second display unit is installed separately from the X-ray imaging apparatus and connected to the X-ray imaging apparatus via a network, and a visual recognition level of the second display unit is better than the visual recognition level of the first display unit.

(Term 2)

The X-ray imaging system according to the Term 1, wherein the second display unit is installed in the surgical operation room in advance and provides at least one function selected from a group consisting of the higher image resolution and the larger display area than the first display unit thereof.

(Term 3)

The X-ray imaging system according to the Term 1 or 2, wherein the foreign object image generation element generates the foreign object image while executing an image processing to enhance the foreign object left inside the subject body after the surgical operation, the X-ray imaging apparatus outputs the foreign object image to the network after the foreign object image generation element generates the foreign object image, and the second display unit displays automatically the foreign object image while the foreign object image output from the X-ray imaging apparatus is forwarded from the network

(Term 4)

The X-ray imaging system according to any one term of the Term 1 to 3, the foreign object image is output from the X-ray imaging apparatus to the network based on an operation for completing an imaging as to the X-ray imaging apparatus.

(Term 5)

The X-ray imaging system according to any one term of the Term 1 to 4, wherein the second display unit further comprises the first display and the second display respectively positioned side by side on the wheeled platform monitor which is parked in advance in the surgical operation room, displays the foreign object image on one display selected from a group consisting the first display and the second display, and one image selected from a group consisting of the X-ray image and the X-ray fluoroscopic image, which is the X-ray fluoroscopy as for the current state of the subject, on the other rest display.

(Term 6)

The X-ray imaging system according to any term of the Term 1 to 4, the second display unit, which is fixed on either the ceiling or the wall of the surgical operation room, displays respectively the X-ray image and the foreign object image at the same time or switches the image between the X-ray image and the foreign object image and then displays the selected image thereon.

(Term 7)

The X-ray imaging system according to any term of the Term 1 to 6, the foreign object left inside the subject body includes at least one material selected from a group consisting of a gauze sponge, a suture needle, and a forceps.

(Term 8)

A foreign malarial recognition method comprises a step of irradiating an X-ray to a subject, a step of detecting the X-ray that transmits through the subject, a step of generating an X-ray image based on detection signals detected thereby, a step of generating a foreign object image to recognize a foreign object left inside the subject body after a surgical operation based on the X-ray image, and a step of displaying the foreign object image on the second display unit, which is installed separately from the X-ray imaging apparatus having the first display unit which displays at least one image from a group consisting of the X-ray image and the foreign object image and is connected to the X-ray imaging apparatus through a network, wherein the better visual recognition level thereof is better than the first display unit thereof.

(Term 9)

The foreign object recognition method according to the term 8, wherein the second display unit is installed in the surgical operation room in advance and has at least one function selected from a group consisting of the higher image resolution and the larger display area than the first display unit thereof.

### REFERENCE OF SIGNS

1 X-ray irradiation unit
2 X-ray detection unit
3 X-ray image generation element
5 Display unit (first display unit)
10 X-ray imaging apparatus
20, 220 Display unit (second display unit)
40 Monitor wheeled platform
41 Foreign object image generation element
51 X-ray image
52 Foreign object image
53 X-ray fluoroscopic image
100, 200, 300 X-ray imaging system
101 Subject
102 Foreign object
110 Network
221 First display
222 Second display

It will be further understood by those of skill in the art that the apparatus and devices and the elements herein, without limitation, and including the sub components such as operational structures, circuits, communication pathways, and related elements, control elements of all kinds, display circuits and display systems and elements, any necessary driving elements, inputs, sensors, detectors, memory elements, processors and any combinations of these structures etc. as will be understood by those of skill in the art as also being identified as or capable of operating the systems and devices and subcomponents noted herein and structures that accomplish the functions without restrictive language or label requirements since those of skill in the art are well versed in related X-Ray Imaging Systems, devices, computer and operational controls and technologies of radiographic devices and all their sub components, including various circuits and combinations of circuits without departing from the scope and spirit of the present invention.

Although only a few embodiments have been disclosed in detail above, other embodiments are possible and the inventors intend these to be encompassed within this specification. The specification describes certain technological solutions to solve the technical problems that are described expressly and inherently in this application. This disclosure describes embodiments, and the claims are intended to cover any modification or alternative or generalization of these embodiments which might be predictable to a person having ordinary skill in the art.

Those of skill would further appreciate that the various illustrative logical blocks, modules, circuits, and algorithm steps described in connection with the embodiments disclosed herein may be implemented as electronic hardware, computer software running on a specific purpose machine that is programmed to carry out the operations described in this application, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the exemplary embodiments.

The various illustrative logical blocks, modules, and circuits described in connection with the embodiments disclosed herein, may be implemented or performed with a general or specific purpose processor, or with hardware that carries out these functions. A general-purpose processor may be a microprocessor, but in the alternative, the processor may be any conventional processor, controller, microcontroller, or state machine. The processor can be part of a computer system that also has an internal bus connecting to cards or other hardware, running based on a system BIOS or equivalent that contains startup and boot software, system memory which provides temporary storage for an operating system, drivers for the hardware and for application programs, disk interface which provides an interface between internal storage device(s) and the other hardware, an external peripheral controller which interfaces to external devices such as a backup storage device, and a network that connects to a hard wired network cable such as Ethernet or may be a wireless connection such as a RF link running under a wireless protocol.

A processor may also be implemented as a combination of computing devices, e.g., a combination of a DSP and a microprocessor, a plurality of microprocessors, one or more microprocessors in conjunction with a DSP core, or any other such configuration. These devices may also be used to select values for devices as described herein.

In one or more exemplary embodiments, the functions described may be implemented in hardware, software, firmware, or any combination thereof. If implemented in software, the functions may be stored on or transmitted over as one or more instructions or code on a computer-readable medium.

Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer.

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

What is claimed is:

1. An X-ray imaging system, comprising:
an X-ray imaging apparatus, further comprising:
an X-ray irradiation element that irradiates an X-ray to a subject;
an X-ray detection element that detects the X-ray that is irradiated from said X-ray irradiation element and transmits through said subject;
an X-ray image generation element that generates an X-ray image based on an X-ray signal detected by said X-ray detection element;
a foreign object image generation element that generates a foreign object image to recognize a foreign object left inside said subject body after a surgical operation based on said X-ray image; and
a first display unit that displays at least one image selected from a group consisting of said X-ray image and said foreign object image;
a second display unit that displays said foreign object image;
wherein said second display unit is installed separately from said X-ray imaging apparatus and connected to said X-ray imaging apparatus via a network; and
wherein a visual recognition level of said second display unit is better than a visual recognition level of said first display unit.

2. The X-ray imaging system, according to claim 1, wherein:
said second display unit that is installed in the surgical operation room provides at least one function selected from a group consisting of a higher image resolution and a larger display area than said first display unit thereof.

3. The X-ray imaging system, according to claim 1, wherein:
said foreign object image generation element generates said foreign object image while executing an image processing to enhance said foreign object left inside said subject body after the surgical operation;
said X-ray imaging apparatus outputs said foreign object image to said network after said foreign object image generation element generates said foreign object image; and
said second display unit displays automatically said foreign object image while said foreign object image output from the said X-ray imaging apparatus is forwarded from said network.

4. The X-ray imaging system, according to claim 1, wherein:
said foreign object image is output from said X-ray imaging apparatus to said network based on an operation for completing an imaging relative to said X-ray imaging apparatus

5. The X-ray imaging system, according to claim 1, wherein:
said second display unit further comprises:
a wheeled platform monitor;
a first display; and
a second display,
wherein said first display and said second display are positioned side by side on said wheeled platform monitor that is parked in advance in a surgical operation room;
wherein one display of said first display and said second display displays said foreign object image, and the other of said first display and said second display displays one image selected from a group consisting of said X-ray image and an X-ray fluoroscopic image that is an X-ray fluoroscopy relative to a current state of said subject.

6. The X-ray imaging system, according to claim 1, wherein:
said second display unit is fixed on either a ceiling or a wall of said surgical operation room and displays respectively said X-ray image and said foreign object image at the same time or one at a time by switching said display image between said X-ray image and said foreign object image.

7. The X-ray imaging system, according to claim 1, wherein:
said foreign object further comprises:
at least one material selected from a group consisting of a gauze sponge, a suture needle and a forceps.

8. A foreign object recognition method, comprising;
a step of irradiating an X-ray to a subject;
a step of detecting the X-ray that transmits through said subject;
a step of generating an X-ray image based on detection signals detected thereby,
a step of generating a foreign object image to recognize a foreign object left inside said subject body after a surgical operation based on said X-ray image, and
a step of displaying said foreign object image on said second display unit, which is installed separately from an X-ray imaging apparatus;
wherein, said X-ray imaging apparatus, further comprises:
said first display unit which displays at least one image from a group consisting of said X-ray image and said foreign object image and is connected to said X-ray imaging apparatus through said network, and
wherein said second display unit provides a better visual recognition level than a visual recognition level of said first display unit.

9. The foreign object recognition method, according to the claim 8, wherein:
said second display unit that is installed in the surgical operation room provides at least one function selected from a group consisting of a higher image resolution and a larger display area than said first display unit provide.
